(19) United States
(12) Patent Application Publication
(10) Pub. No.: US 2017/0369395 A1
(43) Pub. Date: Dec. 28, 2017

(54) ISOPARAFFIN-OLEFIN ALKYLATION

(71) Applicant: ExxonMobil Research and Engineering Company, Annandale, NJ (US)

(72) Inventors: Jihad M. DAKKA, Whitehouse Station, NJ (US); Matthew S. METTLER, Somerville, NJ (US); Jeffrey M. FITT, The Woodlands, TX (US); Charles M. SMITH, Princeton, NJ (US); Ivy D. JOHNSON, Lawrenceville, NJ (US); Christopher M. DEAN, Spring, TX (US); Ajit B. DANDEKAR, Clinton, NJ (US)

(21) Appl. No.: 15/610,734

(22) Filed: Jun. 1, 2017

Related U.S. Application Data
(60) Provisional application No. 62/353,684, filed on Jun. 23, 2016.

(51) Int. Cl. C07C 2/58; B01J 29/70

(52) U.S. Cl. CPC C07C 2/58; B01J 29/70; C07C 2529/70

(57) ABSTRACT

A process for the catalytic alkylation of an olefin with an isoparaffin is described in which a feed comprising at least one olefin and at least one isoparaffin is contacted with a solid acid catalyst under alkylation conditions effective for reaction between the olefin and the isoparaffin to produce an alkylated product. The solid acid catalyst comprises a crystalline microporous material of the MWW framework type, the feed comprises at least one C₅+ olefin and/or at least one C₅+ isoparaffin and the alkylated product comprises at least 20% wt % of C₁₀+ branched paraffins.

### CROSS-REFERENCE TO RELATED APPLICATIONS

This application claims the benefit of U.S. Provisional Application No. 62/353,684, filed on Jun. 23, 2016, the entire contents of which is incorporated herein by reference.

### FIELD

The present disclosure relates to a process for isoparaffin-olefin alkylation.

### BACKGROUND

Alkylation is a reaction in which an alkyl group is added to an organic molecule. Thus an isoparaffin can be reacted with an olefin to provide an isoparaffin of higher molecular weight. Industrially, the concept depends on the reaction of a C₂ to C₅ olefin, normally 2-butene, with isobutane in the presence of an acidic catalyst to produce a so-called alkylate. This alkylate is a valuable blending component in the manufacture of gasoline due not only to its high octane rating but also to its sensitivity to octane-enhancing additives.

Industrial isoparaffin-olefin alkylation processes have historically used hydrofluoric or sulfuric acid catalysts under relatively low temperature conditions. The sulfuric acid alkylation reaction is particularly sensitive to temperature, with low temperatures being favored to minimize the side reaction of olefin polymerization. Acid strength in these liquid acid catalyzed alkylation processes is preferably maintained at 88 to 94 weight percent by the continuous addition of fresh acid and the continuous withdrawal of spent acid. The hydrofluoric acid process is less temperature sensitive and the acid is more easily recovered and purified.

A general discussion of sulfuric acid alkylation can be found in a series of three articles by L. F. Albright et al., “Alkylation of Isobutane with C₄ Olefins”, 27 Ind. Eng. Chem. Res., 381-397, (1988). For a survey of hydrofluoric acid catalyzed alkylation, see 1 Handbook of Petroleum Refining Processes 23-28 (R. A. Meyers, ed., 1986). An overview of the entire technology can be found in “Chemistry, Catalysts and Processes of Isoparaffin-Olefin Alkylation Actual Situation and Future Trends, Corma et al., Catal. Rev.—Sci. Eng. 35(4), 483-570 (1993).

Both sulfuric acid and hydrofluoric acid alkylation share inherent drawbacks including environmental and safety concerns, acid consumption, and sludge disposal. In addition, hydrofluoric and sulfuric acids suffer from the problem that neither is an effective catalyst for alkylation of higher (C₅ and above) olefins and isoparaffins and so the ability to produce C₁₀₊ distillate blending stocks using these catalysts is limited.

Research efforts have, therefore, been directed to developing alkylation catalysts which are equally as effective as sulfuric or hydrofluoric acids but which avoid many of the problems associated with these two acids. In particular, research has been focused on the development of solid, instead of liquid, acid alkylation catalyst systems.

For example, U.S. Pat. No. 3,644,565 discloses alkylation of a paraffin with an olefin in the presence of a catalyst comprising a Group VIII noble metal present on a crystalline aluminosilicate zeolite having pores of substantially uniform diameter from about 4 to 18 angstrom units and a silica to alumina ratio of 2.5 to 10, such as zeolite Y. The catalyst is pretreated with hydrogen to promote selectivity.

However, the development of a satisfactory solid acid replacement for hydrofluoric and sulfuric acid has proved challenging. For example, U.S. Pat. No. 4,384,161 describes a process of alkylating isoparaffins with olefins to provide alkylate using a large-pore zeolite catalyst capable of absorbing 2,2,4-trimethylpentane, for example, ZSM-4, ZSM-20, ZSM-3, ZSM-18, zeolite Beta, faujasite, mordenite, zeolite Y and the rare earth metal-containing forms thereof, and a Lewis acid such as boron trifluoride, antimony pentafluoride or aluminum trichloride. The addition of a Lewis acid is reported to increase the activity and selectivity of the zeolite, thereby effecting alkylation with high olefin space velocity and low isoparaffin/olefin ratio. According to the '161 patent, problems arise in the use of solid catalysts alone in that they appear to age rapidly and cannot perform effectively at high olefin space velocity.

As new solid acid catalysts have become available, they have been routinely screened for their efficacy in isoparaffin-olefin alkylation. For example, U.S. Pat. No. 5,304,698 describes a process for the catalytic alkylation of an olefin with an isoparaffin comprising contacting an olefin-containing feed with an isoparaffin-containing feed with a crystalline microporous material selected from the group consisting of MCM-22, MCM-36, and MCM-49 under alkylation conversion conditions of temperature at least equal to the critical temperature of the principal isoparaffin component of the feed and pressure at least equal to the critical pressure of the principal isoparaffin component of the feed.

Despite extensive research, there remains an unmet need for an improved isoparaffin-olefin alkylation process that is catalyzed by a solid acid catalyst but approaches or exceeds the activity and stability of existing liquid phase processes and can be employed to selectively produce C₁₀₊ distillate blending stocks.

### SUMMARY

According to the present disclosure, it has now been found that MWW framework-type zeolites exhibit unexpectedly high activity and selectivity as isoparaffin-olefin catalysts as well as enhanced flexibility for using different olefins/isoparaffins for producing alkylated products of different carbon number for different applications. In particular, one of the major advantages of MWW framework-type zeolites is their flexibility for handling heavier (C₅₊) olefins and/or isoparaffins to produce C₁₀₊ hydrocarbons when market demand for distillate is high. Moreover, the catalysts show higher stability than existing hydrofluoric and sulfuric acid catalysts, especially when formulated in the absence of amorphous alumina binders.

Thus, in one aspect, the present disclosure provides a process for the catalytic alkylation of an olefin with an isoparaffin, the process comprising: contacting a feed comprising at least one olefin and at least one isoparaffin with a solid acid catalyst under alkylation conditions effective for reaction between the olefin and the isoparaffin to produce an alkylated product, wherein the solid acid catalyst comprises a crystalline microporous material of the MWW framework type, wherein the feed comprises at least one C₅₊ olefin and/or at least one C₅₊ isoparaffin and wherein the alkylated product comprises in excess of 20% wt % of C₁₀₊ branched paraffins.

In a further aspect, the present disclosure provides an isoparaffin-olefin alkylation process for producing alkylated products of different carbon number, the process comprising:

(a) during a first time period, contacting a first feed comprising at least one olefin and at least one isoparaffin with a solid acid catalyst under alkylation conditions effective for reaction between the olefin and the isoparaffin to produce a first alkylated product, wherein the solid acid catalyst comprises a crystalline microporous material of the MWW framework type, wherein the first feed comprises at least one C₅₊ olefin and/or at least one C₅₊ isoparaffin and wherein the first alkylated product comprises in excess of 20% wt % of C₁₀₊ branched paraffins; and

(b) during a second time period, contacting a second feed comprising at least one C₃₊ olefin and at least one C₄₊ isoparaffin with a solid acid catalyst under alkylation conditions effective for reaction between the olefin and the isoparaffin to produce a second alkylated product, wherein the solid acid catalyst comprises a crystalline microporous material of the MWW framework type, and wherein the second alkylated product comprises less than or equal to 20% wt % of C₁₀₊ branched paraffins.

### BRIEF DESCRIPTION OF THE DRAWINGS

FIG. 1 is a graph of isooctene conversion against time on stream (days) for the MCM-49 catalyst of Example 1 in the alkylation of a premixed isobutane/isooctene feed at various temperatures according to the process of Example 2.

FIG. 2 is a graph of % production of 2,2,4-trimethylpentane against total trimethylpentane production for the MCM-49 catalyst of Example 1 in the alkylation of a premixed isobutane/isooctene feed at various temperatures according to the process of Example 2.

FIG. 3 is a graph of butene conversion against material balance (MB) number for an REX catalyst and the MCM-49 catalyst of Example 1 in the alkylation of a premixed isobutane/butene feed according to the process of Example 3.

FIG. 4 provides simulated distillation curves for the liquid products of the alkylation of an isobutane/isooctene feed over the MCM-49 catalyst of Example 1 according to the process of Example 2 and for the liquid products of the alkylation of an isobutane/butene feed over the MCM-49 catalyst of Example 1 according to the process of Example 3.

### DETAILED DESCRIPTION OF THE EMBODIMENTS

As used herein, the term “Cₙ” compound (olefin or paraffin) wherein n is a positive integer, e.g., 1, 2, 3, 4, 5, etc, means a compound having n number of carbon atom(s) per molecule. The term “Cₙ₊” compound wherein n is a positive integer, e.g., 1, 2, 3, 4, 5, etc, means a compound having at least n number of carbon atom(s) per molecule. The term “Cₙ₋” compound wherein n is a positive integer, e.g., 1, 2, 3, 4, 5, etc, as used herein, means a compound having no more than n number of carbon atom(s) per molecule.

Disclosed herein is a process for isoparaffin-olefin alkylation, in which an olefin-containing feed is contacted with an isoparaffin-containing feed under alkylation conditions in the presence of a solid acid catalyst comprising a crystalline microporous material of the MWW framework type. The combined feeds include at least one C₅₊ olefin and/or at least one C₅₊ isoparaffin and surprisingly it is found that the MWW framework type is both active and selective for conversion of these heavier feeds into an alkylated product comprising in excess of 20% wt % of C₁₀₊ branched paraffins useful as a distillate blending stock. In addition, this activity is retained over prolonged periods especially when the catalyst is substantially free of amorphous alumina binder.

In some embodiments, operation of the process with at least one C₅+ olefin and/or at least one C₅+ isoparaffin is periodically alternated with operation with lighter feeds, in which C₃₊ olefins and C₄₊ isoparaffins are converted to produce gasoline blending stocks. Again, the MWW framework type molecular sieve is found to exhibit unusual activity and selectivity thereby allowing flexibility in the carbon number of the alkylated product according to market demand.

As used herein, the term “crystalline microporous material of the MWW framework type” includes one or more of:
molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, the entire content of which is incorporated as reference);
molecular sieves made from a common second degree building block, being a 2-dimensional tiling of such MWW framework topology unit cells, forming a monolayer of one unit cell thickness, preferably one c-unit cell thickness;
molecular sieves made from common second degree building blocks, being layers of one or more than one unit cell thickness, wherein the layer of more than one unit cell thickness is made from stacking, packing, or binding at least two monolayers of MWW framework topology unit cells. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Crystalline microporous materials of the MWW framework type include those molecular sieves having an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

Examples of crystalline microporous materials of the MWW framework type include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in International Patent Publication No. WO97/17290), MCM-36 (described in U.S. Pat. No. 5,250,277), MCM-49 (described in U.S. Pat. No. 5,236,575), MCM-56 (described in U.S. Pat. No. 5,362,697), UZM-8 (described in U.S. Pat. No. 6,756,030), UZM-8HS (described in U.S. Pat. No. 7,713,513), UZM-37 (described in U.S. Pat. No. 7,982,084); EMM-10 (described in U.S. Pat. No. 7,842,277), EMM-12 (described in U.S. Pat. No. 8,704,025), EMM-13 (described in U.S. Pat. No. 8,704,023), MIT-1 (described by Luo et al in Chem. Sci., 2015, 6, 6320-6324), and mixtures thereof, with MCM-49 generally being preferred.

In some embodiments, the crystalline microporous material of the MWW framework type employed herein may be an aluminosilicate material having a silica to alumina molar ratio of at least 10, such as at least 10 to less than 50.

In some embodiments, the crystalline microporous material of the MWW framework type employed herein may be contaminated with other crystalline materials, such as ferrierite or quartz. These contaminants may be present in quantities ≦10% by weight, normally ≦5% by weight.

The above molecular sieves may be used in the alkylation catalyst without any binder or matrix, i.e., in so-called self-bound form. Alternatively, the molecular sieve may be composited with another material which is resistant to the temperatures and other conditions employed in the alkylation reaction. Such materials include active and inactive materials and synthetic or naturally occurring zeolites as well as inorganic materials such as clays and/or oxides such as alumina, silica, silica-alumina, zirconia, titania, magnesia or mixtures of these and other oxides. The latter may be either naturally occurring or in the form of gelatinous precipitates or gels including mixtures of silica and metal oxides. Clays may also be included with the oxide type binders to modify the mechanical properties of the catalyst or to assist in its manufacture. Use of a material in conjunction with the molecular sieve, i.e., combined therewith or present during its synthesis, which itself is catalytically active may change the conversion and/or selectivity of the catalyst. Inactive materials suitably serve as diluents to control the amount of conversion so that products may be obtained economically and orderly without employing other means for controlling the rate of reaction. These materials may be incorporated into naturally occurring clays, e.g., bentonite and kaolin, to improve the crush strength of the catalyst under commercial operating conditions and function as binders or matrices for the catalyst. The relative proportions of molecular sieve and inorganic oxide binder may vary widely. For example, the amount of binder employed may be as little as 1 wt %, such as at least 5 wt %, for example at least 10 wt %, whereas in other embodiments the catalyst may include up to 90 wt %, for example up 80 wt %, such as up to 70 wt %, for example up to 60 wt %, such as up to 50 wt % of a binder material.

In one embodiment, the solid acid catalyst employed in the present process is substantially free of any binder containing amorphous alumina. As used herein, the term “substantially free of any binder containing amorphous alumina” means that the solid acid catalyst used herein contains less than 5 wt %, such as less than 1 wt %, and preferably no measurable amount, of amorphous alumina as a binder. Surprisingly, it is found that when the solid acid catalyst is substantially free of any binder containing amorphous alumina, the activity of the catalyst for isoparaffin-olefin alkylation can be significantly increased, for example by at least 50%, such as at least 75%, even at least 100% as compared with the activity of an identical catalyst but with an amorphous alumina binder.

The present alkylation process is suitably conducted at temperatures from about 275° F. to about 700° F. (135° C. to 371° C.), such as from about 300° F. to about 600° F. (149° C. to 316° C.). Operating temperatures typically exceed the critical temperature of the principal component in the feed. The term “principal component” as used herein is defined as the component of highest concentration in the feedstock. For example, isobutane is the principal component in a feedstock consisting of isobutane and 2-butene in an isobutane:2-butene weight ratio of 50:1.

Operating pressure may similarly be controlled to maintain the principal component of the feed in the supercritical state, and is suitably from about 300 to about 1500 psig (2170 kPa-a to 10,445 kPa-a), such as from about 400 to about 1000 psig (2859 kPa-a to 6996 kPa-a). In some embodiments, the operating temperature and pressure remain above the critical value for the principal feed component during the entire process run, including the first contact between fresh catalyst and fresh feed.

Hydrocarbon flow through the alkylation reaction zone containing the catalyst is typically controlled to provide a total liquid hourly space velocity (LHSV) sufficient to convert about 99 percent by weight of the fresh olefin to alkylate product. In some embodiments, olefin LHSV values fall within the range of about 0.01 to about 10 hr⁻¹.

The present isoparaffin-olefin alkylation process can be conducted in any known reactor, including reactors which allow for continuous or semi-continuous catalyst regeneration, such as fluidized and moving bed reactors, as well as swing bed reactor systems where multiple reactors are oscillated between on-stream mode and regeneration mode. Surprisingly, however, it is found that catalysts employing MWW framework type molecular sieves show unusual stability when used in isoparaffin-olefin alkylation even with feeds containing C₅₊ olefins and/or C₅₊ isoparaffins. Thus, MWW-containing alkylation catalysts are particularly suitable for use in simple fixed bed reactors, without swing bed capability. In such cases, cycle lengths (on-stream times between successive catalyst regenerations) in excess of 150 days may be obtained.

Feedstocks useful in the present alkylation process include at least one isoparaffin and at least one olefin. The isoparaffin reactant used in the present alkylation process may have from about 4 to about 8 carbon atoms. Representative examples of such isoparaffins include isobutane, isopentane, 3-methylhexane, 2-methylhexane, 2,3-dimethylbutane, 2,4-dimethylhexane and mixtures thereof. In some embodiments, mixtures of isoparaffins may be employed, such as a mixture of isobutane and isopentane, where the weight ratio of isobutane to isopentane may range from 1:9 to 9:1.

The olefin component of the feedstock may include at least one olefin having from 2 to 12 carbon atoms. Representative examples of such olefins include butene-2, isobutylene, butene-1, propylene, ethylene, hexene, heptene and octene, merely to name a few. In some embodiments, the olefin component of the feedstock is selected from the group consisting of propylene, butenes, pentenes and mixtures thereof. For example, in one embodiment, the olefin component of the feedstock may include a mixture of propylene and at least one butene, especially 2-butene, where the weight ratio of propylene to butene is from 0.01:1 to 1.5:1, such as from 0.1:1 to 1:1. In another embodiment, the olefin component of the feedstock may include a mixture of propylene and at least one pentene, where the weight ratio of propylene to pentene is from 0.01:1 to 1.5:1, such as from 0.1:1 to 1:1.

Isoparaffin to olefin ratios in the reactor feed typically range from about 1.5:1 to about 100:1, such as 10:1 to 75:1, measured on a volume to volume basis, so as to produce a high quality alkylate product at industrially useful yields. Higher isoparaffin:olefin ratios may also be used, but limited availability of produced isoparaffin within many refineries coupled with the relatively high cost of purchased isoparaffin favor isoparaffin:olefin ratios within the ranges listed above.

The olefin-containing feedstock and the isoparaffin-containing feedstock may be mixed prior to being fed to the alkylation reaction zone or may be supplied separately to the reaction zone. In addition, before being sent to the alkylation reaction zone, the isoparaffin and/or olefin may be treated to remove catalyst poisons e.g., using guard beds with specific absorbents for reducing the level of S, N, and/or oxygenates to values which do not affect catalyst stability activity and selectivity.

During at least part of the operation of the present process, the feedstock comprises at least one C₅₊ olefin and/or at least one C₅₊ isoparaffin such that the alkylated product comprises in excess of 20 wt %, such as at least 30 wt %, such as at least 50 wt %, even up to 90 wt %, of C₁₀₊ branched paraffins. Typically, the resultant alkylated product contains less than 20 wt % of C₁₆ to C₂₀ hydrocarbons and less than 5 wt % of C₂₀ to C₂₅ hydrocarbons. In some embodiments, the C₁₀₊ alkylated product has a cetane number in excess of 30 and the C₉₋ alkylated product has octane number in excess of 85. Thus, by separating, for example by distillation, all or a fraction of the C₁₀₊ component of the alkylated product, it is possible to recover a blending stock suitable for combining with a refinery distillate pool.

In one embodiment, the feedstock during at least part of the process operation comprises at least one C₅₊ olefin, such as at least one C₅ to C₁₂ olefin, for example isooctene, and at least one C₄₊ isoparaffin, especially isobutene. In the latter case, the alkylated product is typically found to comprise at least 10 wt %, for example at least 30 wt %, such as at least 60 wt % of 2,3,4 and 2,3,3 and 2,2,4-trimethylpentane, an excellent blending stock for a refinery gasoline pool.

In another embodiment, the feedstock during at least part of the process operation comprises at least one C₃₊ olefin, such as at least one butene or a mixture of propylene and at least one butene, and at least one C₅₊ isoparaffin.

In some embodiments, the present process is continuously operated with the feedstock comprising at least one heavy (C₅+) olefinic or isoparaffinic component. However, in other embodiments, it may be desirable to periodically shift between a first mode of operation with a first feedstock comprising at least one C₅₊ component and a second mode of operation with a second feedstock comprising at least one C₂₊ olefin and at least one C₄₊ isoparaffin. In the second mode of operation, the alkylated product typically comprises about 20 wt % of C₅-C₇ hydrocarbons, 60-65 wt % of octanes and 15-20 wt % of C₉+ hydrocarbons. At least part of the C₈₋ paraffin-containing fraction can then be separated from the alkylated product for blending with a refinery gasoline pool. Depending on the time of year and the demand for gasoline versus distillate, the process can be tuned to produce the desired alkylated product.

The invention will now be more particularly described with reference to the following non-limiting Examples and the accompanying drawings.

### Example 1 Preparation of 80 wt % MCM-49/20 wt % Alumina Catalyst

80 parts MCM-49 zeolite crystals are combined with 20 parts pseudoboehmite alumina, on a calcined dry weight basis. The MCM-49 and pseudoboehmite alumina dry powder are placed in a muller or a mixer and mixed for about 10 to 30 minutes. Sufficient water and 0.05% polyvinyl alcohol are added to the MCM-49 and alumina during the mixing process to produce an extrudable paste. The extrudable paste is formed into a 1/20 inch quadralobe extrudate using an extruder. After extrusion, the 1/20th inch quadralobe extrudate is dried at a temperature ranging from 250° F. to 325° F. (121 to 163° C.). After drying, the dried extrudate is heated to 1000° F. (538° C.) under flowing nitrogen. The extrudate is then cooled to ambient temperature and humidified with saturated air or steam.

After humidification, the extrudate is ion exchanged with 0.5 to 1 N ammonium nitrate solution. The ammonium nitrate solution ion exchange is repeated. The ammonium nitrate exchanged extrudate is then washed with deionized water to remove residual nitrate prior to calcination in air. After washing the wet extrudate, it is dried. The exchanged and dried extrudate is then calcined in a nitrogen/air mixture to a temperature 1000° F. (538° C.).

### Example 2 Testing of Example 1 Catalyst in Isobutane/Isooctene Alkylation

The catalyst of Example 1 was used in the alkylation testing of a model feed comprising a mixture of isobutane and isooctene having the following composition by weight:

| | |
|---|---|
| iso-C₈ = | 2.4% |
| iso-butane | 97.37% |
| n-butane | 0.23% |

The reactor used in these experiments comprised a stainless steel tube having an internal diameter of ⅜ in, a length of 20.5 in and a wall thickness of 0.035 in. A piece of stainless steel tubing 8¾ in. long×⅜ in. external diameter and a piece of ¼ inch tubing of similar length were positioned in the bottom of the reactor (one inside of the other) as a spacer to position and support the catalyst in the isothermal zone of the furnace. A ¼ inch plug of glass wool was placed at the top of the spacer to keep the catalyst in place. A ⅛ inch stainless steel thermo-well was placed in the catalyst bed, long enough to monitor temperature throughout the catalyst bed using a movable thermocouple. The catalyst is loaded with a spacer at the bottom to keep the catalyst bed in the center of the furnace's isothermal zone.

The catalyst was then loaded into the reactor from the top. The catalyst bed typically contained about 4 gm of catalyst sized to 14-25 mesh (700 to 1400 micron) and was 10 cm. in length. A ¼ in. plug of glass wool was placed at the top of the catalyst bed to separate quartz chips from the catalyst. The remaining void space at the top of the reactor was filled with quartz chips. The reactor was installed in the furnace with the catalyst bed in the middle of the furnace at the pre-marked isothermal zone. The reactor was then pressure and leak tested typically at 300 psig (2170 kPa-a).

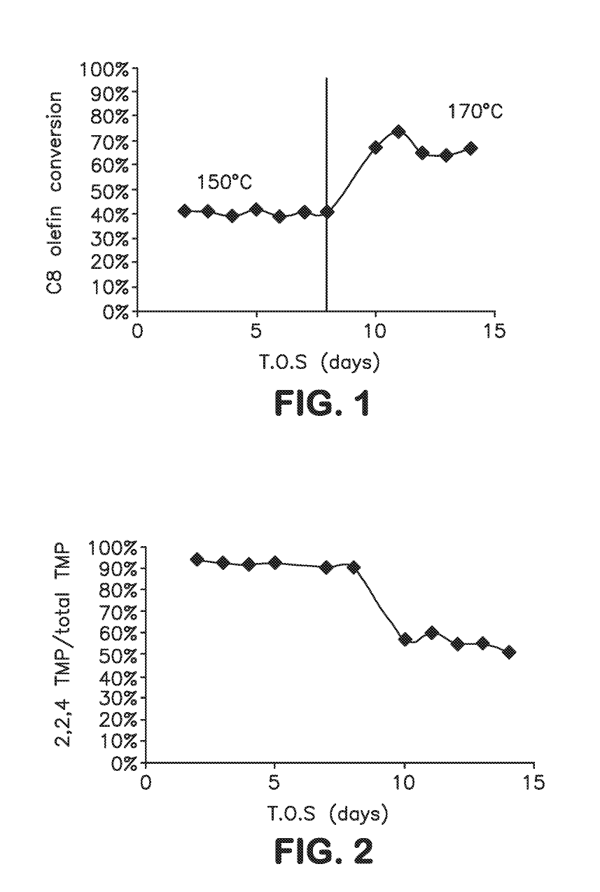

500 cc ISCO syringe pumps were used to introduce the feed to the reactor. Two ISCO pumps were used for pumping the iso-butane (high flow rate 50-250 cc/hr) and one ISCO pump for pumping isooctene (1-5 cc/hr). A Grove “Mity Mite” back pressure controller was used to control the reactor pressure typically at 750 psig (5272 kPa-a). On-line GC analyses were taken to verify feed and the product composition. The feeds were then pumped through the reactor with the temperature initially being held at 150° C. and then, after eight days on stream, increased to 170° C. The products exiting the reactor flowed through heated lines routed to GC then to three cold (5-7° C.) collection pots in series. The non-condensable gas products were routed through a gas pump for analyzing the gas effluent. Material balances were taken at 24 hr intervals. Samples were taken for analysis. The material balance and the gas samples were taken at the same time while an on-line GC analysis was conducted for doing material balance. The results of the catalytic testing are summarized in FIGS. 1 and 2.

FIG. 1 shows that the C₈₌ conversion remained substantially constant at around 40% during the first eight days on stream at 150° C. and then increased to around 60-70% when the temperature was increased to 170° C. and then again stayed constant at this higher range for the remaining five days of the test.

FIG. 2 shows that the 2,2,4-dimethylpentane selectivity remained substantially constant at around 90% during the first eight days of the test then decreased to around 60% when the temperature was increased from 150 to 170° C. The 2,2,4-dimethylpentane selectivity showed some further small decrease during the final five days at 170° C. It is to be appreciated that the majority of the trimethyl pentane is formed by alkylation of isobutane in the feed with isobutylene formed by hydride transfer between the isobutane and isooctene in the feed. These results not only show that heavy (C5+) olefins can be used as alkylating agents over the MWW zeolite, but also these olefins can undergo hydride transfer with isoparaffins to generate the corresponding isoolefins which can further alkylate the isoparaffin feed to produce high octane products, such as 2,2,4-dimethylpentane.

### Example 3 Testing of Example 1 Catalyst and REX in Isobutane/Isobutene Alkylation

The process of Example 2 was repeated but with the catalyst being either the MCM-49/alumina catalyst of Example 1 or REX and the feed being a mixture of isobutane and 2-butene having the following composition by weight:

| | |
|---|---|
| 1-butene | 0.01% |
| Cis-2-butene | 1.25% |
| Trans-2-butene | 1.19% |
| Iso-C₄ = | 0.00% |
| Iso-butane | 97.37% |
| n-butane | 0.23% |

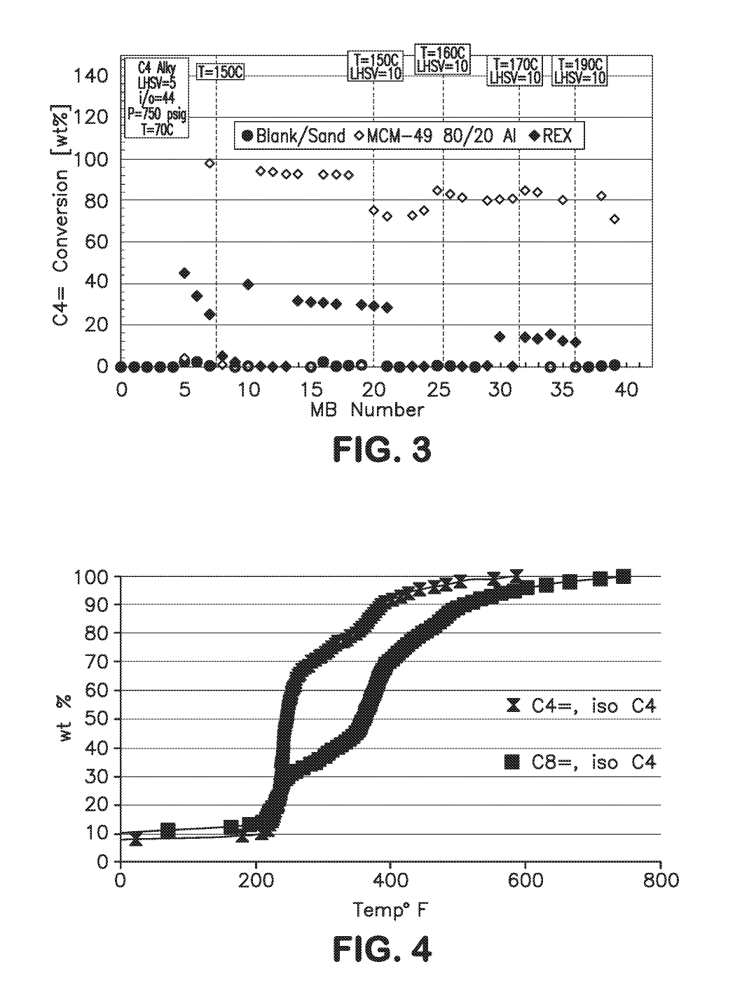

The results are shown in FIG. 3 and, when compared with the data in FIG. 1, demonstrate that the REX catalyst was less active and deactivated more rapidly in butene alkylation with isobutane than the corresponding properties of the MCM-49 catalyst in octene alkylation with isobutane.

FIG. 4 compares simulated distillation curves for the liquid products of the alkylation of the isobutane/isooctene feed according to the process of Example 2 and for the liquid products of the alkylation of the isobutane/butene feed according to the process of Example 3, with the catalyst in each case being the MCM-49/alumina catalyst of Example 1. The data clearly shows that a higher concentration of heavy product is formed when C₈= was used instead of C₄= in the alkylation of isobutane. These heavy products can be used either as diesel or kerosene. This approach provides a way to convert heavy olefins to fluids, diesel and kerosene using alkylation chemistry over a heterogeneous catalyst.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A process for the catalytic alkylation of an olefin with an isoparaffin, the process comprising: contacting a feed comprising at least one olefin and at least one isoparaffin with a solid acid catalyst under alkylation conditions effective for reaction between the olefin and the isoparaffin to produce an alkylated product, wherein the solid acid catalyst comprises a crystalline microporous material of the MWW framework type, wherein the feed comprises at least one C₅₊ olefin and/or at least one C₅₊ isoparaffin and wherein the alkylated product comprises in excess of 20% wt % of C₁₀₊ branched paraffins.

2. The process of claim 1, wherein the feed comprises at least one C₅ to C₁₂ olefin and at least one C₄₊ isoparaffin.

3. The process of claim 2, wherein the feed comprises iso-octene.

4. The process of claim 2, wherein the feed comprise isobutene and the alkylated product comprises at least 10 wt % of 2,2,4-trimethylpentane.

5. The process of claim 1, wherein the feed comprises at least one C₃₊ olefin and at least one C₅ to C₈ isoparaffin.

6. The process of claim 1, wherein the feed comprises isobutane and isopentane.

7. The process of claim 1, wherein the alkylated product contains less than 5 wt % of C₂₀ to C₂₅ hydrocarbons.

8. The process of claim 1, wherein the alkylated product contains less than 20 wt % of C₁₆ to C₂₀ hydrocarbons.

9. The process of claim 1, wherein the C₁₀₊ alkylated product has a cetane number of at least 30.

10. The process of claim 1, wherein the solid acid catalyst is substantially binder-free.

11. The process of claim 1, wherein the solid acid catalyst comprises an inorganic oxide binder.

12. The process of claim 11, wherein the inorganic oxide binder comprises alumina.

13. The process of claim 11, wherein the inorganic oxide binder is substantially free of amorphous alumina.

14. The process of claim 11, wherein the inorganic oxide binder comprises silica.

15. The process of claim 5, wherein the crystalline microporous material of the MWW framework type is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, MIT-1, and mixtures thereof.

16. The process of claim 1, wherein the crystalline microporous material of the MWW framework type comprises MCM-49.

17. The process of claim 1, wherein the MWW framework type material contains up to 10% by weight of impurities of other framework structures.

18. The process of claim 1, wherein the alkylation conditions include a temperature at least equal to the critical temperature of the principal component of the combined olefin-containing feed and isoparaffin-containing feed and pressure at least equal to the critical pressure of the principal component of the combined olefin-containing feed and isoparaffin-containing feed.

19. An isoparaffin-olefin alkylation process for producing alkylated products of different carbon number, the process comprising:
(a) during a first time period, contacting a first feed comprising at least one olefin and at least one isoparaffin with a solid acid catalyst under alkylation conditions effective for reaction between the olefin and the isoparaffin to produce a first alkylated product, wherein the solid acid catalyst comprises a crystalline microporous material of the MWW framework type, wherein the first feed comprises at least one C₅₊ olefin and/or at least one C₅₊ isoparaffin and wherein the first alkylated product comprises in excess of 20% wt % of C₁₀₊ branched paraffins; and
(b) during a second time period, contacting a second feed comprising at least one C₂₊ olefin and at least one C₄₊ isoparaffin with a solid acid catalyst under alkylation conditions effective for reaction between the olefin and the isoparaffin to produce a second alkylated product, wherein the solid acid catalyst comprises a crystalline microporous material of the MWW framework type, and wherein the second alkylated product comprises less than or equal to 20% wt % of C₁₀₊ branched paraffins.

20. The process of claim 19 and further comprising:
(c) separating a C₁₀₊ branched paraffin-containing fraction from the first alkylated product; and
(d) blending at least part of the C₁₀₊ branched paraffin-containing fraction with a refinery distillate pool.

21. The process of claim 17 and further comprising:
(e) separating a C₉₋ paraffin-containing fraction from at least one of the first alkylated product and the second alkylated product; and
blending at least part of the or each C₉₋ paraffin-containing fraction with a refinery gasoline pool.

22. A hydrocarbon product produced by isoparaffin-olefin alkylation and comprising at least 20 wt % of a C₁₀₊ fraction having a cetane number in excess of 30 and a C₉₋ fraction having an octane number in excess of 85.

23. The hydrocarbon product of claim 22 and comprising at least 50 wt % of the C₁₀₊ fraction.

24. The hydrocarbon product of claim 22 and comprising less than 20 wt % of C₁₆ to C₂₀ hydrocarbons and less than 5 wt % of C₂₀ to C₂₅ hydrocarbons.
